(19) United States
(12) Patent Application Publication
(10) Pub. No.: US 2005/0031714 A1
(43) Pub. Date: Feb. 10, 2005

(54) Psyllium

(75) Inventor: Thomas E. Gray, Green Bay, WI (US)

Correspondence Address: VENABLE, BAETJER, HOWARD AND CIVILETTI, LLP, P.O. BOX 34385, WASHINGTON, DC, 20043-9998, US

(73) Assignee: Enzymatic Therapy, Inc., Green Bay, WI (US)

(21) Appl. No.: 10/702,608

(22) Filed: Nov. 7, 2003

Related U.S. Application Data
(60) Provisional application No. 60/492,740, filed on Aug. 6, 2003.

(51) Int. Cl. A61K 35/78

(52) U.S. Cl. 424/738

(57) ABSTRACT

A coated psyllium powder, which uniformly disperses in an aqueous medium, is prepared by mixing the powder with glycerin. The psyllium is present in an amount of at least 90% by weight and has particle size between 20 to 200 mesh. The amount of glycerin is between 2 and 10% by weight.

### BACKGROUND OF THE INVENTION

Psyllium seed husk has been used as a fiber supplement and a bulk laxative drug because the psyllium seed husk has a capacity for substantial swelling when ingested. This swelling makes the psyllium husk a useful bulking agent. Ingestion of psyllium seed husk has been demonstrated to yield a number of health benefits to the consumer, including normalizing bowel function, appetite suppression and cholesterol reduction.

The psyllium seed includes a husk portion that is a cleaned, dried seed coat of the psyllium seed. The husk portion is separated from the seed by winnowing and thrashing. Typically, the husk portion of the psyllium seed is ground into a powder to make the bulk laxative agent.

Psyllium seed husk has hydrophobic characteristics due to natural waxes and oils on the surface of the seed coat, leaf, husk, etc. Without rigorous mixing it forms a gelatinous mass on contact with water, and it exhibits poor dispersibility and wettability in water. The psyllium husk particles tend to agglomerate when mixed with water. Hydration takes place over the surface of such agglomerated aggregates to form gel-coated lumps, the interiors of which are still substantially dry. These lumps are extremely difficult to disperse. It is often desirable to increase the dispersability or wettability of these products to enhance rapid integration with minimum agitation and the avoidance of clumps that resist dissolution.

A number of attempts have been made in the past to improve the dispersability and/or wettability of psyllium. The attempts include controlling particle size of the psyllium powder during a processing step of size reduction. A product is described in U.S. Pat. No. 4,996,051 that includes apple fiber, fructose, gum arabic, flavors and psyllium husk powder having a particle size that passes through a No. 50 mesh screen.

Cold blending has been used to improve the dispersibility of psyllium husk powder. It involves mixing psyllium powder with granular soluble diluents such as sucrose and dextrose. Other granular diluents include chemical compounds capable of reacting when in contact with moisture to produce carbon dioxide. The granular diluents are added in concentrations of up to 50% of the cold blend.

The cold blending of psyllium with granular diluents has created a situation where the more dense diluent will fall to the bottom of a container of water while the psyllium powder will ball up near or on the surface of the water. Moreover, the solubility of the granular diluents is much higher than the psyllium husk powder. This increased solubility favors rapid dispersibility, and more importantly, dissolution of the granular diluents. However, the psyllium powder is left behind in an undispersed state.

Another problem of cold blending psyllium powder with a granular diluent such as sucrose or dextrose is that these diluents are metabolizable sugars. They are not acceptable to diabetic or caloric sensitive consumers.

The coextrusion of psyllium husks with citric acid under controlled heating conditions has also been used to improve the dispersibility of psyllium husk powder. The coextrusion is believed to effect a reduction in microbial growth and to improve dispersibility.

A process is described in U.S. Pat. No. 4,321,263 for coating granulated psyllium particles with an alcoholic solution of either polyethylene glycol (PEG) or polyvinyl pyrrolidone (PVP) and granulating the coated particles. The use of PVP and PEG is, however, limited by handling problems resulting from high viscosities developed in PVP and PEG solutions. Furthermore, the use of volatile alcohol to deliver PEG, PVP or blends of PEG and PVP to psyllium powder surfaces during processing has presented safety problems to many processors because of the flammable nature of alcohol.

A process is described in U.S. Pat. Nos. 4,459,280 and 4,548,806 for improving mixability and dispersibility of psyllium mucilloid by applying a film of hydrolyzed starch oligosaccharide, either a mono- or di-saccharide, a polyglucose, or a polymaltose to the psyllium.

A process that includes the blending of psyllium with and food grade emulsifiers followed by solvent removal is described in U.S. Pat. No. 4,551,331. The solvents includes ethyl alcohol, water, ethyl alcohol and ethyl acetate and mixtures thereof. The emulsifiers include distilled propylene glycol monoglycerides, distilled monoglycerides and sodium stearyl lactylate, hydrophilic ethoxylated sorbitan monoesters, malto dextran, lecithin, and mono- and diglycerides.

A coated fibrous, vegetable material is described in U.S. Pat. No. 4,828,842. The coating includes a combination of a major amount of hydroxypropyl methylcellulose and a minor amount of polyethylene glycol to aid in the wetting and dispersing of the fibrous, vegetable material.

A tablet is described in U.S. Pat. No. 4,999,200 that includes psyllium powder, a gelling agent such as polysorbate 80, a binding agent such as polyvinyl pyrollidone or acacia and a disintegrant such as microcrystalline cellulose. The tablet is described as disintegrating in the gastrointestinal tract after ingestion.

The use of an edible acid dispersant is disclosed in U.S. Pat. No. 5,219,570. The edible acid dispersant is described as occurring throughout the agglomerating coating applied to psyllium husk. The coating improves mixability, dispersibility, and product aesthetics for psyllium husk products having low (less than about 20%) sugar content.

A method to coat psyllium with gum arabic is described in U.S. Pat. No. 6,312,730. The coated psyllium particles disperse rapidly in the water.

A method is described in U.S. Pat. No. 6,337,048 reduces the hydrophobic characteristics of psyllium husk by fractionating the surface waxes or oils using a gaseous mixture containing 01, 02 and 03 (hereinafter referred to as “Ox”). This is done without damaging the gums or the cellular structure. The gas mixture is applied to the material in a sealed chamber. However, this method requires the use of equipment not generally available to most manufactures.

While there has already been much research devoted to improving the solubility and dispersibility of products such as psyllium in liquids, there has not been much research to improve the disintegration of psyllium in capsule form. The objective of the present invention is to provide a psyllium powder in capsule form with quick disintegration and without agglomeration.

It is also desirable that the psyllium powder be sugar-free so that it is acceptable for use in diabetics and/or people on a restricted sugar diet and also be easier to use, e.g. avoid the need for premixing.

### SUMMARY OF THE INVENTION

The present invention is directed to a psyllium powder with glycerin as a coating agent to enable its quick and uniform dispersability in an aqueous medium and to methods for its preparation. The coated psyllium particles are suitable for use in capsules. The coated psyllium particles can disintegrate rapidly and thoroughly in a patient's gastrointestinal tract. Because this composition does not have any sugar additives like dextrose, it is more acceptable for diabetics and/or people on a restricted sugar diet.

### DETAIL DESCRIPTION OF THE INVENTION

The psyllium husk used in the present invention is from psyllium seeds, from plants of the Plantago genus. Various species such as Plantago lanceolata, P. rugelii, and P. major are known. Commercial psyllium husk include the French (black; Plantago U.S. Pat. No. 6,337,048 describes a indica), Spanish (P. psyllium) and Indian (blonde; P. ovata). Indian (blonde) psyllium husk is preferred for use herein. Also preferred is psyllium husk which is 95% pure or at least 85 to 90% pure. Compositions of the present invention are comprised of psyllium husk from about 25% to 99%, preferably from 50% to about 98%, and more preferably from about 50% to about 90%. It is also preferred that the psyllium husk used herein have reduced particle size. Preferably the particle size of the psyllium husk is such that more than about 90% of the psyllium husk will pass through a 40 mesh screen, and more preferably such that essentially all will pass through an 80 mesh screen. The density of a suitable psyllium powder loose is approximately 0.44 grams per cubic centimeter. The same material has a tapped density of 0.50 grams per cubic centimeter.

Glycerin, also known as glycerol and glycerine, is a clear, colorless, odorless, viscous, hygroscopic liquid. It is miscible with water and has been widely used in pharmaceutical formulas, including oral, ophthalmic, and parental preparations. Glycerin aids dispersion of the psyllium because of its hydrophobic nature—by slowing down the hydration and swelling of psyllium so it does not clump upon wetting/hydration.

Previous inventions mentioned above use polysorbate 80 or other wetting agents. They might improve the disintegration of psyllium in capsule form without agglomeration. In contrast, the invention disclosed herein impedes wettability by employing hydrophobic glycerin, thereby allowing dispersion to occur. Dissipation of the glycerin from the surface of the psyllium particle after disintegration will then permit hydration. Once particles are separated after disintegration, wetting and swelling will occur but the psyllium will avoid gel formation/binding since the individual particles are no longer closely packed. Furthermore, this is so effective that it works in hot water, where the tendency for gelation would be even more pronounced were it not for the dispersing benefits of glycerin.

This process has major advantages over competitive psyllium capsules, which do not have good disintegration/dispersion properties. This process obviates the need to pre-mix psyllium in water prior to consumption. It is more convenient because it is easily portable. There is a reduced tendency to cause esophageal/intestinal blockage, since disintegration is much enhanced. This is a product for anyone desiring the convenience of a no-mix psyllium supplement deliverable in capsule form.

For psyllium to exert its health benefits, it must be fully dispersed in the intestines. A series of dispersion experiments were conducted on untreated psyllium. It was found that the gelatin capsule disintegrated, and the fluid wet the exterior of the psyllium contained in the capsule. This wetting caused the external surface psyllium granules to bind closely with its adjacent granules of untreated psyllium, creating a tightly bound outer wet mucoidal shell that encapsulated the internal untreated psyllium granules and prevented the water from penetrating to the interior, creating a psyllium lump. This lump did not dissolve, even after 24 hours, but continued to hydrate allowing water to penetrate into the non-dispersing lump when held static. Since the psyllium is not fully released from the capsule, impeded by the above described process, it cannot exert full benefits in the human intestine performing as a fiber supplement.

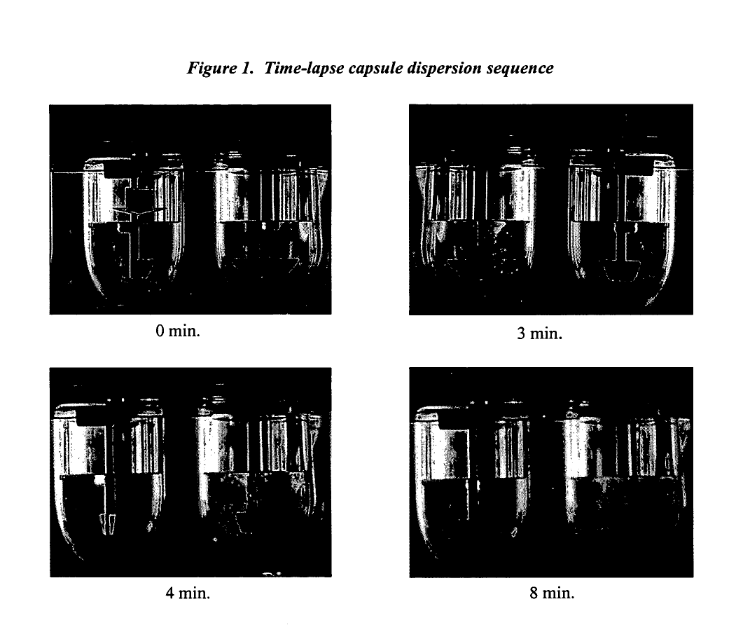

The invention results in the uniform dispersion of psyllium powder upon capsule rupture, because of a glycerin coating that allows uniform wetting of the psyllium powder without clumping. This in turn facilitates uniform dispersability of the psyllium in the intestine, after swallowing the capsule, as simulated in FIG. 1, to accomplish an increased mode of absorption and action.

### EXAMPLE

To coat the psyllium powder, glycerin solution is mixed with the psyllium powder at a ratio of 2 to 10% glycerin to 90 to 98% psyllium powder by weight.

### Dispersion Time in 0.1N HCl Solution

Method: 500 milligrams of coated psyllium powder was encapsulated into a clear gelatin size 00 capsule. A control containing 500 milligrams of uncoated psyllium powder was encapsulated in the same manner. The capsules were immersed into a room temperature (approximately 70° F.) degassed 0.1N HCl solution and agitated with paddles revolving at 75 RPM. See FIG. 1 for time-lapse photo dispersion sequence. The coated psyllium capsule is in the chamber on the right side of each photo and the uncoated psyllium capsule is in the chamber on the left side of each photo. An arrow points to the uncoated capsule in the upper left time 0 photo. The coated psyllium capsule's gelatin exterior dissolved and began to disperse after 3 to 4 minutes with complete dispersion achieved within 10 minutes. The uncoated psyllium capsule's gelatin exterior dissolved leaving a psyllium lump that did not completely disperse even after 2 hours of agitation.

1. A composition comprising psyllium and an effective amount of a coating agent to uniformly disperse the psyllium in an aqueous medium, wherein the coating agent is glycerin.

2. The composition according to claim 1 wherein the psyllium is present in an amount of at least 90% by weight.

3. The composition according to claim 1 wherein the psyllium is a powder having 20 to 200 mesh particle size.

4. The composition according to claim 3 wherein the mesh particle size is less than 80 mesh.

5. The composition according to claim 1 wherein the psyllium is a powder having a density of approximately 0.44 grams per cubic centimeter.

6. The composition of claim 1 wherein the psyllium is a powder having a tapped density of 0.50 grams per cubic centimeter.

7. The composition according to claim 1 wherein the effective amount of glycerin is 2 to 10% glycerin by weight.

8. The composition according to claim 1 wherein the composition is in a capsule.

9. A method of coating psyllium powder comprising
mixing a psyllium powder with an effective amount of glycerin to uniformly coat the psyllium particles so that the resultant coated powder is uniformly disperse in an aqueous medium.

10. A method of claim 9 wherein the effective amount is between 2-10% glycerin by weight.

11. The method according to claim 9 wherein the psyllium powder has 20 to 200 mesh particle size.

12. The method according to claim 9 wherein the psyllium powder has a particle size less than 80 mesh.

13. The method of claim 9 wherein the psyllium powder has a density of approximately 0.44 grams per cubic centimeter.

14. The method of claim 9 wherein the psyllium powder has a tapped density of 0.50 grams per cubic centimeter.

15. The method of claim 9 wherein the resultant coated powder is formed or place into a capsule.

16. The coated psyllium powder prepared by the method of claim 9.
